(19) United States
(12) Patent Application Publication
(10) Pub. No.: US 2023/0139704 A1
(43) Pub. Date: May 4, 2023

(54) OPTICAL FIBER FIXTURES FOR IMAGING DEVICES

(71) Applicant: Lumicell, Inc., Newton, MA (US)

(72) Inventors: Michael Bush, Arlington, MA (US); Joseph D'Anello, Braintree, MA (US); Steven Cappetta, Sudbury, MA (US)

(73) Assignee: Lumicell, Inc., Newton, MA (US)

(21) Appl. No.: 17/977,241

(22) Filed: Oct. 31, 2022

Related U.S. Application Data
(60) Provisional application No. 63/275,728, filed on Nov. 4, 2021.

(51) Int. Cl. A61B 1/00; A61B 1/04

(52) U.S. Cl. CPC A61B 1/00165; A61B 1/043

(57) ABSTRACT

Embodiments related to medical imaging devices including rigid imaging tips and their methods of use for identifying abnormal tissue within a surgical bed are disclosed. An imaging device may include a housing having a first channel and a light guide disposed at least partially in the first channel. The imaging device may also include a clamp disposed in the housing, where the clamp is configured to apply a force to a rigid exterior portion of the light guide in a clamp direction transverse to the light guide longitudinal axis to secure the light guide to the housing. The clamp may have a clamp longitudinal axis parallel to a light guide longitudinal axis.

### RELATED APPLICATIONS

This Application claims the benefit of priority under 35 U.S.C. § 119(e) of U.S. Provisional Application Ser. No. 63/275,728, filed Nov. 4, 2021, the disclosure of which is incorporated herein by reference in its entirety.

### FIELD

Disclosed embodiments are related to optical fiber fixtures for medical imaging devices and related methods of use.

### BACKGROUND

There are over one million cancer surgeries per year performed in the United States and nearly 40% of them miss resecting the entire tumor according to the National Cancer Institute Surveillance Epidemiology and End Results report. Residual cancer in the surgical bed is a leading risk factor for local tumor recurrence, reduced survival rates and increased likelihood of metastases. In a typical solid tumor resection, the surgeon removes the bulk of the tumor and sends it to pathology. The pathologist then samples the bulk tumor in a few locations and images a stained section under a microscope to determine if the surgeon has completely removed all of cancer cells from the patient. Should the pathologist find a portion of the stained sample with cancer cells bordering ink (a diagnostic known in the medical realm as “positive margin”), the surgeon may be instructed to resect more tissue. However, this pathology exercise is a time intensive procedure and often takes days for final results to be sent to the physician. Should a pathology report requiring additional resection return after the patient has completed the initial surgery, this may require the surgeon to perform a second surgery.

Some conventional surgical methods include employing fluorescent imaging devices. The imaging devices may employ one or more imaging agents configured to bind or otherwise be retained in cancerous or other abnormal tissue. The one or more imaging agents may fluoresce when exposed to an excitation light. In some cases, an imaging device may detect the presence of the fluorescent agent, thereby indicating the presence of additional cancerous or other abnormal tissue to remove during the surgical method.

### SUMMARY

In some embodiments, an imaging device includes a housing including a first channel, and a light guide disposed at least partially in the first channel, where the light guide includes a rigid exterior portion on a distal end portion of the light guide, and where the light guide has a light guide longitudinal axis. The imaging device also includes a clamp disposed in the housing, where the clamp is configured to apply a force to the rigid exterior portion of the light guide in a clamp direction transverse to the light guide longitudinal axis to secure the light guide to the housing.

In some embodiments, an imaging device includes a housing including a first channel, and a light guide disposed at least partially in the first channel, where the light guide includes a rigid exterior portion on an exterior distal end portion of the light guide, and wherein the light guide has a light guide longitudinal axis. The imaging device also includes a clamp disposed in the housing, where the clamp has a clamp longitudinal axis parallel to the light guide longitudinal axis, and where the clamp is configured to apply a force to the rigid exterior portion to secure the light guide to the housing.

In some embodiments, a method of assembling an imaging device includes positioning a light guide in a first channel of a housing, adjusting a longitudinal position of the light guide in the housing from a first position to a second position, and applying force to a rigid exterior portion of the light guide with a clamp to secure the light guide to the housing in the second position.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic representation of a surgical bed being imaged with decreased magnification;

FIG. 2 is a perspective view of one embodiment of a handheld medical imaging device;

FIG. 3 is a partially exploded view of one embodiment of a probe of a handheld medical imaging device;

FIG. 4A is a side cross-sectional view taken along line 4A-4A of FIG. 3;

FIG. 4B is a perspective cross-sectional view taken along line 4A-4A of FIG. 3;

FIG. 5 is a perspective exploded view of one embodiment of a light guide clamp;

FIG. 6A is a rear view of the light guide clamp of FIG. 5;

FIG. 6B is rear view of a clamp body of the light guide clamp of FIG. 6A;

FIG. 7A is a side view of the light guide clamp of FIG. 5 in a first state;

FIG. 7B is a side view of the light guide clamp of FIG. 5 in a second state;

FIG. 8 is a side view of one embodiment of an imaging device housing including a light guide clamp;

FIG. 9 is a cross-sectional view of the imaging device housing of FIG. 8 taken along line 9-9;

FIG. 10A is a schematic of one embodiment of a focus of a light guide at a focal plane of an imaging device;

FIG. 10B is a schematic of another embodiment of a focus of a light guide at a focal plane of an imaging device;

FIG. 11 is a flow chart for one embodiment of assembling an imaging device;

FIG. 12 is a side view of one embodiment of a tapered housing portion; and

FIG. 13 is a front view of the tapered housing portion of FIG. 12.

### DETAILED DESCRIPTION

According to exemplary embodiments described herein, a medical imaging device may be employed to detect the presence of abnormal tissue during a surgical procedure, such as removal of cancerous cells or other abnormal cells. The optical arrangement of the imaging device may be important for the ability for the imaging device to detect the presence of abnormal tissue. For example, for fluorescent imaging, factors that may affect the ability of the imaging device to detect abnormal cells include, but are not limited to, an excitation light intensity and uniformity at a focal plane of the imaging device. The inventors have appreciated that these factors may be based at least in part on a spacing between optical components of the imaging device. The inventors have especially appreciated that relative spacing between a fiber optic light guide and other optical components is important to provide the desired or necessary excitation light intensity for performing certain imaging techniques including, for example, fluorescence imaging and identification of abnormal tissue in a surgical field or other portion of a subject's body. For example, in instances in which a fluorescent imaging device identifies abnormal tissue based on a threshold fluorescence intensity, an excitation light intensity that is less than expected may result in correspondingly lower fluorescence intensities, which may result in not detecting abnormal tissue located within the field of view of the imaging device. An appropriate relative spacing between the fiber optic light guide and other optical components (e.g., mirrors, lenses, etc.) may allow for an appropriate focus and light intensity of light emitted from the fiber optic light guide that reaches a surgical tissue bed or other portion of a subject's body. The inventors have also appreciated that conventional methods of fixing a fiber optic light guide in a housing are difficult, inaccurate, and/or risk damaging the sensitive optical fibers which may also lead to light scattering and reduced excitation light intensities reaching the tissue being imaged.

In view of the above, the inventors have appreciated that the reliable positioning of a fiber optic light guide in a housing may be desirable for appropriate functioning of an imaging device. In particular, the inventors have appreciated the benefits of systems and methods as described herein for adjusting a position of a light guide in a housing relative to other optical components and fixing the light guide in an appropriate position for the functioning of the imaging device. The systems and methods of exemplary embodiments described herein may allow for the adjustment of an optical spacing of the light guide to compensate for a tolerance stack of optical components between the light guide and a distal end portion of an imaging device where the light from the light guide exits. The systems and methods of exemplary embodiments described herein may also allow a desired focus of light from the light guide to be provided at a target focal plane or within a depth of field of the imaging device. The systems and methods of exemplary embodiments described herein may also allow light intensities within a predetermined tolerance of a predetermined light intensity (e.g., excitation light for a fluorescent agent) to be provided to a target surgical bed. In some embodiments, systems and methods according to exemplary embodiments described herein may include a clamp configured to fix a light guide in a desired position relative to the housing and other optical components of the imaging device.

In some conventional imaging devices, a light guide may be oriented perpendicular to a handle of an imaging device, which is oftentimes parallel to a longitudinal axis of an imaging device. Such an arrangement may be due to a desire to address the challenges discussed above with regards to spacing between optical components. An arrangement with a perpendicularly oriented light guide may have fewer optical components between the light guide and a distal end portion and/or a focal plane of the imaging device where the light from the light guide exits and it may be easier to position an end of a light guide, such as a fiber optic cable, in a desired position in such an arrangement. However, such an arrangement may be bulky and inconvenient to handle. Additionally, such an arrangement may complicate positioning of the device near a surgical tissue bed, routing of a light guide cable, and stress management in light guides such as fiber optic cables.

Accordingly, in view of the above, the inventors have appreciated the benefits of an imaging device that is ergonomic and easy to manipulate in a surgical environment. In particular, the inventors have appreciated the benefits of an imaging device having a light guide having a light guide longitudinal axis that is at least partially parallel with a portion of an optical path of the imaging device. For example, for an imaging device having an optical path parallel to a longitudinal axis of the imaging device, at least a portion of the light guide longitudinal axis may also be parallel to a longitudinal axis of the imaging device. In this manner, the light guide may not extend transversely (e.g., perpendicularly) away from the imaging device, thereby facilitating the manipulation of the imaging device. In some embodiments, the light guide may extend at least partially through a housing of the imaging device. For example, the light guide may extend through a proximal portion of a housing of the imaging device. In such an arrangement, the light guide may be parallel to the optical path in the proximal portion of the housing.

In some embodiments, an imaging device includes a housing and a light guide disposed at least partially in the housing. The light guide may be disposed in a proximal portion of the housing. The imaging device may have an optical path which passes from a proximal portion of the housing and through a distal end portion and/or focal plane of the imaging device. In some embodiments, the light guide has a light guide longitudinal axis that is parallel to the optical path through a proximal portion of the housing. In some embodiments, at least a portion, including a distal portion, of the light guide longitudinal axis may be parallel to a longitudinal axis of the imaging device housing. In some embodiments, the light guide may be disposed at least partially in a first channel and may be adjustable (e.g., slidable) between multiple positions along the light guide longitudinal axis in the first channel. The imaging device may include a clamp configured to apply force to the light guide to secure the light guide to the housing and fix the position and orientation of the light guide relative to the housing. In some embodiments, the light guide includes a rigid exterior portion on a distal end portion of the light guide. The clamp may be configured to apply force to the rigid exterior portion, and the rigid exterior portion may protect the light guide from damage by substantially shielding the light guide from the applied clamping force. For example, the rigid exterior portion may protect one or more optical fibers of the light guide from being crushed and/or broken. In some embodiments, the clamp has a clamp longitudinal axis parallel to the light guide longitudinal axis.

In some embodiments, an imaging device includes a clamp configured to secure the light guide to the housing. In particular, the clamp may be configured to fix the position and orientation of the light guide in the housing, such that a relative position between the light guide and other optical components of the imaging device may also be fixed. The inventors have appreciated the benefits of a clamp that is simple to adjust and that may be compact within a housing. Additionally, the inventors have appreciated the benefits of a clamp that applies force to fix a light guide without damaging or breaking the light guide. In some embodiments, the clamp may be configured to apply force to the light guide in a clamp direction transverse to the light guide longitudinal axis to secure the light guide to the housing. For example, in some embodiments, the clamp direction may be perpendicular relative to an associated portion of the light guide longitudinal axis the clamp interfaces with. In some embodiments, the light guide may be disposed in a first channel of the housing, and the clamp may be disposed in a second channel of the housing. The light guide may extend from within the second channel to the first channel. The clamp may be configured to engage the light guide in the second channel in the clamp direction. In some embodiments, the first channel has a first channel axis, the second channel has a second channel axis parallel to the first channel axis though other arrangements and orientations may also be used. In some embodiments, the clamp may be adjustable to fix or release the light guide in an adjustment direction parallel to the light guide longitudinal axis.

According to exemplary embodiments described herein, a clamp may be configured to apply force to a light guide (e.g., in a clamp direction). In some cases, a light guide may be fragile or otherwise sensitive to externally applied forces. For example, a fiber optic light guide may be susceptible to breakage of internal optical fibers if too much force is applied to the light guide. For example, fiber optic light guides are especially susceptible to damage from applied shear stresses. Accordingly, in some embodiments, a light guide may include a rigid exterior portion configured to support a majority, and in some instances substantially all, of a clamping force applied by the clamp. In some embodiments, the rigid exterior portion is configured as a tubular covering for a cylindrical light guide. In some embodiments, the rigid exterior portion is a jacket. The rigid exterior portion may be formed of metal, rigid plastic, or another suitable material. The rigid exterior portion may be configured to resist a clamping force applied by the clamp, such that a portion of the light guide protected by the rigid exterior portion is not damaged. In some embodiments, the rigid exterior portion may be disposed on a distal end portion of a light guide. The distal end portion of the light guide may be received in a housing of an imaging device and may be disposed adjacent to, and/or within, the clamp. In some embodiments, a light guide may include a rigid portion configured to project the light guide from damage by the clamping force of the clamp. In such an embodiment, the light guide may include a rigid shell or other structure formed from a sufficiently rigid metal, plastic, or other suitable material configured to protect the internal components of the light guide. Of course, any suitable protective arrangement may be employed for a light guide, as the present disclosure is not so limited.

In some embodiments, a clamp of an imaging device may be configured to secure a light guide in an imaging device housing by applying a clamping force in a clamp direction transverse to a longitudinal axis of the light guide. In some embodiments, the clamp may include a clamp body, a clamp wedge, and an adjustment fastener. The adjustment fastener may secure the clamp body to the clamp wedge. The adjustment fastener may also be configured to adjust a longitudinal spacing between the clamp body and the clamp wedge. The clamp wedge may be configured to move transverse to the light guide longitudinal axis when the longitudinal spacing between the clamp body and the clamp wedge is adjusted by the adjustment fastener. For example, in some embodiments, a clamp wedge includes a wedge inclined surface, and the clamp body includes a body inclined surface engaged with the wedge inclined surface. Accordingly, when the spacing between the clamp wedge and clamp body is adjusted, the wedge may move transversely relative to the clamp body as the wedge moves along the body inclined surface. According to exemplary embodiments described herein, the body inclined surface and the wedge inclined surface may be inclined relative to a longitudinal axis of the clamp. In some embodiments, the angle may be between 15 and 75 degrees, 30 and 60 degrees, 30 and 45 degrees, and/or any other appropriate range of angles. Of course, any suitable angle may be employed, as the present disclosure is not so limited. Additionally, while some embodiments herein are described as having two inclined surfaces, in other embodiments a single inclined surface may be employed for a clamp, as the present disclosure is not so limited. For example, in some embodiments, a camming surface (e.g., a non-inclined camming surface) may be employed that engages the single inclined surface. In some embodiments, the camming surface may not have a complementary inclination and/or shape relative to the single inclined camming surface. However, the camming surface may nevertheless engage the single inclined surface to move a portion of a clamp transversely relative to a clamp longitudinal axis.

According to exemplary embodiments described herein, an adjustment fastener may include a threaded shaft threadedly engaged with a clamp wedge and disposed in a slot of a clamp body. In such embodiments, rotation of the threaded shaft may move the clamp wedge toward or away from the clamp body (e.g., along a longitudinal axis of the clamp), thereby also moving the clamp wedge transversely. In some embodiments, an adjustment fastener may be a bolt disposed in a hole of the clamp wedge and a slot of the clamp body and secured with a nut. In such embodiments, rotation of the nut may move the clamp wedge toward or away from the clamp body (e.g., along a longitudinal axis of the clamp), thereby also moving the clamp wedge transversely. Of course, any suitable adjustment fastener may be employed, including a screw, bolt, or other fastener arrangement, as the present disclosure is not so limited. In some embodiments, an adjustment fastener of a clamp may be configured to move with a transversely moving component of the clamp (e.g., a clamp wedge). In some such embodiments, a clamp body may include a slot configured to allow the adjustment fastener to move transversely relative to a light guide longitudinal axis.

In some embodiments, a clamp of an imaging device may be configured to apply a clamp force to a light guide, which may yield a maximum pressure applied to the light guide. In some embodiments, the clamp may include one or more grooves configured to spread the force over an area of the light guide to reduce the maximum pressure applied to the one or more external surfaces of the light guide. For example, in some embodiments, the one or more grooves may have a shape complementing a shape of the light guide. That is, the one or more grooves may have a shape that receives and fits the form of at least a portion of the light guide. For example, in some embodiments a distal portion of a light guide may be cylindrical. According to this example, the one or more grooves may have a complementary semi-cylindrical shape configured to receive the cylindrical distal portions therein. Due to the complementary shapes of the distal portion and the one or more grooves, force applied through the interface between the distal portion and the one or more grooves may be spread across the distal portion, rather than being concentrated in a smaller region (e.g., a point or a line). Of course, while a particular set of corresponding shapes is described, other appropriate shapes for the light guide and the grooves may also be used as the disclosure is not so limited.

According to exemplary embodiments described herein, a light guide may be movable between multiple positions in a housing and may be fixed in a position with a clamp. In some embodiments, the light guide may be movable a suitable distance to compensate for a tolerance stack associated with a plurality of optical components included in the imaging device during manufacture, such that a desired focal plane for light emitted from the light guide may be achieved. Moreover, the position of the light guide may be adjustable such that a desired illumination intensity may be achieved at the desired focal plane. In some embodiments, a light guide may be movable within an imaging device housing along a longitudinal axis of the light guide by a distance between 0.01 mm and 0.5 mm, 0.1 mm and 1 mm, 0.1 mm and 2 mm, 1 mm and 15 mm, 2 mm and 5 mm, 5 mm and 10 mm, 2.5 mm and 12 mm and/or any other appropriate distance. Of course, a light guide may be adjustable any suitable distance within a housing, as the present disclosure is not so limited. In some embodiments, moving the light guide within an imaging device housing may correspondingly move a focal plane of the light emitted from the light guide. The inventors have appreciated that in some cases it may be desirable to defocus light emitted from the light guide relative to a focal plane of the imaging device so as to provide a more uniform illumination at a target tissue bed. Defocusing may be especially desirable in cases where the light guide includes optical fibers, which may otherwise produce a narrow-focused light. In some embodiments, defocusing light emitted from the light guide may include moving the light guide from a first position in the housing to a second position in the housing. In some embodiments, defocusing light emitted from the light guide includes dealigning (e.g., making non-overlapping) a focal plane of the light guide and a focal plane of a photosensitive detector of an imaging device.

As discussed above, the inventors have appreciated that in some cases it may be desirable to defocus light emitted from the light guide relative to a focal plane of the imaging device so as to provide a more uniform illumination at a target tissue bed. Accordingly, in some embodiments, a focus of a light guide may be located at a plane offset by a predetermined distance from a focal plane of the imaging device. In some embodiments, the predetermined distance of the offset may be between 0.01 mm and 0.5 mm, 0.1 mm and 0.5 mm, 0.25 mm and 1 mm, 0.5 mm and 2 mm, 1 mm and 5 mm, 1 mm and 10 mm, and/or any other appropriate distance. Such offsets may provide defocused light at the focal plane of the imaging device to more uniformly illuminate the field of view of the imaging device compared with focused light from the light guide. In some embodiments, the focus of the light guide may be adjustable by moving a light guide within a housing along a light guide longitudinal axis, as discussed above.

According to exemplary embodiments described herein, a light guide may be configured to provide a desired illumination intensity to a target tissue bed. As discussed above, movement of a light guide within a housing between multiple positions may change an illumination intensity ultimately emitted onto the target tissue bed. Accordingly, in some embodiments, a light guide may be moved within a housing to provide the desired illumination intensity. In particular, in some embodiments, a light guide may be moved within a housing and then fixed in a position where a desired illumination intensity is achieved. In some embodiments, a light guide may be moved to provide an illumination intensity between about 10 mW/cm² to 200 mW/cm² at a desired focal plane for imaging tissue within a surgical bed, though other illumination intensities might also be used. For example, an illumination intensity of 50 mW/cm² to 200 mW/cm², 100 mW/cm² to 200 mW/cm², 150 mW/cm² to 200 mW/cm², and/or any other appropriate intensity for a desired application could also be used in other embodiments.

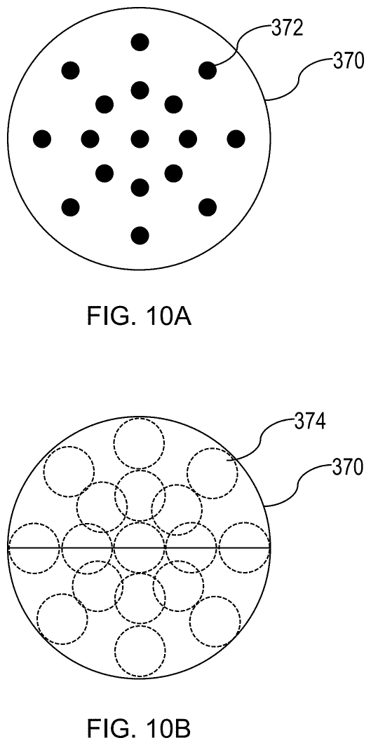

In some embodiments, a method of assembling an imaging device includes positioning a light guide in a housing (e.g., in a first channel). In some embodiments, positioning the light guide in the first channel includes orienting the light guide toward a mirror disposed in the housing. The mirror may be configured to reflect light from the light guide. In some embodiments, light may be reflected from the light guide off the mirror in a direction transverse to a light guide longitudinal axis. In some embodiments, light may be reflected from the light guide off the mirror toward a dichroic mirror disposed in the housing. The method may also include adjusting a longitudinal position of the light guide in the housing from a first position to a second position. In some embodiments, in the first position light from the light guide may be focused on a focal plane aligned with a distal end portion (e.g., a distal end) of the imaging device. In some embodiments, in the second position the light from the light guide may be defocused on the distal end portion (e.g., a distal end) of the imaging device. Accordingly, in some embodiments, moving the light guide from the first position to the second position may include moving a focal plane of the light emitted from the light guide. For example, moving the light guide from the first position to the second position may include moving a focal plane from a first focal plane position aligned with a distal end of the imaging device to a second focal plane position that is not aligned with the distal end of the imaging device. Such an arrangement may improve the uniformity of illumination by light emitted from the light guide, as will be discussed further below with reference to the exemplary embodiment of FIGS. 10A-10B. In some embodiments, the method may also include applying force to the light guide with a clamp to secure the light guide to the housing in the second position. In some embodiments, applying force to the light guide may include applying force to a rigid exterior portion disposed on a distal end portion of the light guide. In some embodiments, applying force to the rigid exterior portion of the light guide includes applying the force in a clamp direction transverse to a light guide longitudinal axis of a portion of the light guide associated with the clamp.

In some embodiments, a method of assembling an imaging device may include positioning one or more optical components in a housing of the imaging device housing. In some embodiments, the one or more optical components may include one or more mirrors, one or more light directing components (e.g., a dichroic mirror), a photosensitive detector, one or more filters, one or more lenses, one or more windows, and/or any other suitable optical components. Various optical components will be described in further detail in reference to exemplary embodiments below. Positioning the one or more optical components may include establishing an optical path of the imaging device between a photosensitive detector and a distal end portion and/or focal plane of the imaging device. The method may also include positioning a light guide in the housing. In some embodiments, positioning the light guide in the housing may include positioning the light guide in a proximal portion of the housing. In some embodiments, positioning the light guide includes making a light guide longitudinal axis parallel to the optical path through the proximal portion of the housing. In some embodiments, the optical path through the proximal portion of the housing may be parallel to the longitudinal axis of the imaging device. The method may also include adjusting a longitudinal position of the light guide in the housing from a first position to a second position. Adjusting the longitudinal housing from the first position to the second position may allow the light guide to compensate for a tolerance stack of the one or more optical components so that a desired focus and intensity of light emitted from the light guide and traveling out of a distal end portion of the imaging device is achieved.

In some embodiments, an imaging device may include a tapered housing portion configured to provide strain relief for a light guide secured in the housing. In some embodiments, the tapered housing may include a strain relief plug configured to fix a proximal portion of the light guide to the housing. In some embodiments, the light guide may be flexible such that a distal end portion of the light guide may be moved relative to the proximal portion fixed by the strain relief plug. In some embodiments, the strain relief plug may be an epoxy plug. Of course, any suitable material for fixing a proximal portion of the light guide relative to the housing may be employed, as the present disclosure is not so limited. In some embodiments, the strain relief plug may be configured to seal a proximal portion of the housing against liquid and/or air ingress.

According to exemplary embodiments described herein, a handheld medical imaging device may be employed to detect the presence of abnormal tissue with an appropriate imaging agent. In some embodiments, the medical imaging device may provide sufficient illumination of an excitation wavelength of the imaging agent to generate a fluorescence signal from the imaging agent that exceeds instrument noise of the imaging device. In some embodiments, the illumination provided by the medical imaging device may also result in an autofluorescence signal from healthy tissue. The medical imaging device may also detect abnormal tissue at sizes ranging from centimeters to sizes on the order of 10 micrometers to tens of micrometers. Other size scales are also possible. As described in more detail below, in some embodiments, it may be desirable for the medical imaging device to be able to image a large field of view in real-time and/or be relatively insensitive to human motions inherent in a handheld device as well as natural motions of a patient involved in certain types of surgery such as breast cancer and lung cancer surgeries. The imaging device may either be used for imaging surgical beds, such as tumor beds, intact tissue surfaces, and/or it may be used for imaging already excised tissue as the disclosure is not so limited.

In one embodiment, a medical imaging device may include a rigid imaging tip including a distal end portion defining a focal plane at a fixed distance from an optically associated photosensitive detector. For example, a distally extending member may define at its distal end a focal plane of the photosensitive detector. Depending on the embodiment, optics associated with the photosensitive detector may either fix a focus of the photosensitive detector at the focal plane located at the distal end of the rigid imaging tip, or they may permit a focus of the photosensitive detector to be shifted between the focal plane located at the distal end of the rigid imaging tip and another focal plane located beyond the distal end of the rigid imaging tip. While any appropriate photosensitive detector might be used, exemplary photosensitive detectors include a charge-coupled device (CCD) detector, a complementary metal-oxide semiconductor (CMOS) detector, and an avalanche photo diode (APD). The photosensitive detector may include a plurality of pixels such that an optical axis passes from the focal plane of the rigid imaging tip to the photosensitive detector.

Depending on the embodiment, a medical imaging device can also include one or more light directing elements for selectively directing light from a light guide including an excitation wavelength of an imaging agent towards a distal end of the device while permitting emitted light including an emission wavelength of the imaging agent to be transmitted to the photosensitive detector. In one aspect, a light emitting element comprises a dichroic mirror positioned to reflect light below a wavelength cutoff towards a distal end of an associated imaging tip while permitting light emitted by the imaging agent with a wavelength above the wavelength cutoff to be transmitted to the photosensitive detector. However, it should be understood that other ways of directing light towards a distal end of the device might be used including, for example, fiber optics located within the rigid tip, and other appropriate configurations.

An imaging device may also include appropriate optics to focus light emitted from within a field of view of the device onto a photosensitive detector with a desired resolution. To provide the desired resolution, the optics may focus the emitted light using any appropriate magnification onto a photosensitive detector including a plurality of pixels. Depending on a size of the individual pixels, the optics may either provide magnification, demagnification, or no magnification as the current disclosure is not so limited. Without wishing to be bound by theory, a typical cancer cell may be on the order of approximately 15 μm across. In some embodiments, an optical magnification of the optics within a medical imaging device may be selected such that a field of view of each pixel may be equal to or greater than about 1 μm, 2 μm, 3 μm, 4 μm, 5 μm, 10 μm, 15 μm, 30 μm, or any other desired size. Additionally, the field of view of each pixel may be less than about 100 μm, 50 μm, 40 μm, 30 μm, 20 μm, 10 μm, or any other desired size scale. In one specific embodiment, the field of view per pixel may be between about 5 μm and 100 μm inclusively. In another embodiment, the field of view per pixel may be between about 5 μm and 50 μm inclusively.

It should be understood that the various embodiments described here may be used with any appropriate type of light guide. However, in some embodiments, the light guide described in the various embodiments disclosed herein may correspond to a fiber optic cable configured to be attached to a separate external light source such that the fiber optic cable transmits the excitation light to the imaging device. Appropriate types of light sources may include, but are not limited to, light emitting diodes, lasers, and/or any other appropriate type of light source. Additionally, the light guide may provide excitation light in any desired range of wavelengths. For example, in one embodiment, a light guide may provide light with wavelengths between or equal to about 300 nm to 1,000 nm, 590 nm to 680 nm, 600 nm to 650 nm, 620 nm to 640 nm, or any other appropriate range of wavelengths depending on the particular imaging agent being used. Depending on the particular imaging agent being used, the various components of the medical imaging device may also be constructed and arranged to collect emission wavelengths from an imaging agent that are about 300 nm to 1,000 nm, 590 nm to 680 nm, 600 nm to 650 nm, 620 nm to 640 nm, or any other appropriate range of wavelengths.

An exemplary imaging agent capable of providing the desired detection depths noted above is pegulicianine (LUM015). LUM015 and its use is further described in U.S. Patent Application Publication No. 2011/0104071 and U.S. Patent Application Publication No. 2014/0301950, which are included herein by references in their entirety. Other appropriate fluorophores that might be included in an imaging agent include, but are not limited to, Cy3, Cy3.5, Cy5, Alexa 568, Alexa 546, Alexa 610, Alexa 647, ROX, TAMRA, Bodipy 576, Bodipy 581, Bodipy TR, Bodipy 630, VivoTag 645, and Texas Red. Of course, one of ordinary skill in the art will be able to select imaging agents with fluorophores suitable for a particular application.

While various combinations of optical components, light guides, and light sources are described above and in reference to the figures below, it should be understood that the various optical components such as filters, dichroic mirrors, fiber optics, mirrors, prisms, and other components are not limited to being used with only the embodiments they are described in reference to. Instead, these optical components may be used in any combination with any one of the embodiments described herein.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

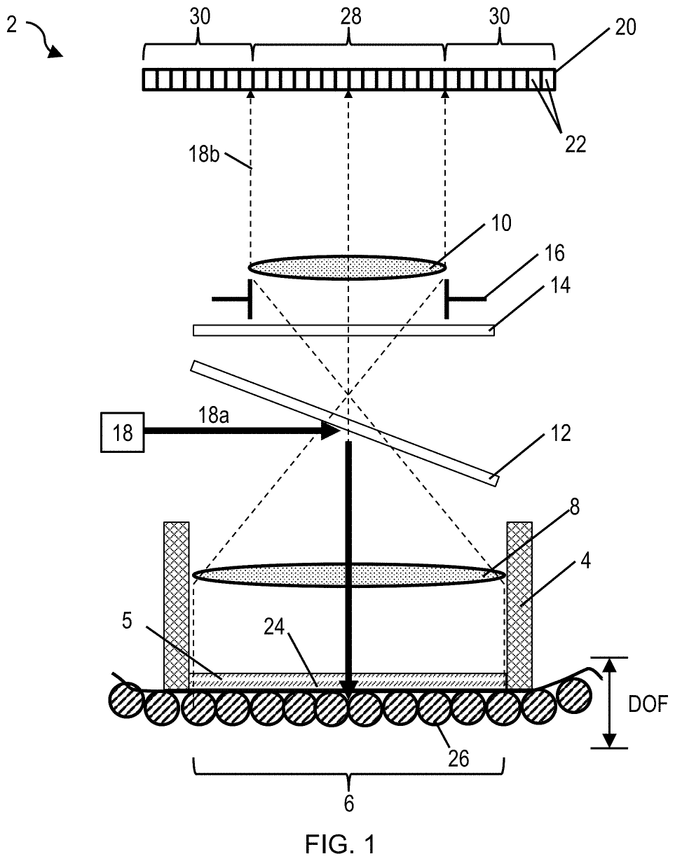

FIG. 1 depicts a schematic representation of exemplary embodiments for components of a medical imaging device 2. The medical imaging device may include a rigid imaging tip 4 at least partially defined by a distally extending member, frustoconical cylinder or other hollow structure. The rigid imaging tip 4 may be constructed and arranged to be held against tissue to fix a focal length of the medical imaging device relative to the tissue. As shown in FIG. 1, the rigid imaging tip includes an optically transparent window 5 that may be pressed into the tissue bed 24 to flatten the tissue at the fixed focal length of the medical imaging device. As depicted in FIG. 1, the rigid imaging tip 4 may also include an opening at a distal end that defines a field of view 6. The medical imaging device 2 may also include optics such as an objective lens 8, an imaging lens 10, and an aperture 16. The optics may focus light emitted from the field of view 6 onto a photosensitive detector 20 including a plurality of pixels 22. The medical imaging device may also include features such as a dichroic mirror 12 and a filter 14. While a doublet lens arrangement has been depicted in FIG. 1, it should be understood that other types of optics capable of focusing the field of view 6 onto the photosensitive detector 20 might also be used including, for example, fiber-optic bundles. Additionally, the photosensitive detector may correspond to any appropriate type of photosensitive detector configured to image or otherwise acquire a light-based signal from the field of view including photosensitive detectors such as a charge-coupled device (CCD), a complementary metal oxide semiconductor (CMOS) array, an avalanche photodiode (APD) array, or other appropriate detector.

As illustrated in FIG. 1, the medical imaging device may be positioned such that a distal end of the rigid imaging tip 4 may be pressed against a surgical bed 24 including one or more cells 26 which may be marked with a desired imaging agent. Instances where all, a portion, or none of the cells are marked with the imaging agent are contemplated. Pressing the rigid tip against the surgical bed may prevent out of plane and lateral tissue motion, which may allow for the use of collection optics with larger f numbers and consequently, larger collection efficiencies, smaller blur radii, and smaller depth of field. Additionally, pressing the rigid imaging tip 4 against the surgical bed may provide a fixed focal length between the tissue bed 24 and photosensitive detector 20. In some embodiments, the rigid imaging tip may have a length such that the distal end of the rigid imaging tip is also located at a focal plane of the photosensitive detector 20 in at least one mode of operation (e.g., when the photosensitive detector is focused on a fixed focal plane defined by the window 5). In some such embodiments, in at least one mode of operation the medical imaging device may have a fixed focal length between the tissue bed 24 and the photosensitive detector 20 as the tissue bed is pressed against the window 5. As shown in FIG. 1, the window 5 may be flat, such that the window flattens the tissue bed 24 into alignment with the distal end of the rigid imaging tip. In some embodiments, the medical imaging device may include a variable focus. According to such embodiments, in at least one mode of operation the focal plane may be adjustable, such that the focus may be set by a user based on the window 5 and tissue bed 24. For example, prior to use of the medical imaging device, the focal plane may be aligned with the window 5, or a position based at least in part on the window. As shown in FIG. 1, pressing the rigid imaging tip against the surgical bed may position the surgical bed 24 and the cells 26 contained therein within a predetermined distance (e.g., within a depth of field (DOF) of the imaging device) of the focal plane of the imaging device.

In some embodiments, it may be desirable to maintain a fixed distance between a distal end of the rigid imaging tip and the photosensitive detector. This may help to maintain the focus of tissue located within the focal plane defined by the distal end of the rigid imaging tip. Therefore, the rigid imaging tip may be adapted to resist deflection and/or deformation when pressed against a surgical bed such that tissue located within the focal plane defined by the distal end of the rigid imaging tip is maintained in focus.

During use, the medical imaging device may be associated with a light source 18 that directs light 18a with a first range of wavelengths towards the dichroic mirror 12. The first range of wavelengths may correspond to an excitation wavelength of a desired imaging agent. In some instances, the light source 18 may include appropriate components to collimate the light 18a. The light source 18 might also include one or more filters to provide a desired wavelength, or spectrum of wavelengths, while filtering out wavelengths like those detected by the photosensitive detector 20. In some embodiments, the dichroic mirror 12 may have a cutoff wavelength that is greater than the first range of wavelengths. Thus, the dichroic mirror 12 may reflect the incident light 18a towards a distal end of the rigid imaging tip 4 and onto the surgical bed 24. When the one or more cells 26 that are labeled with a desired imaging agent are exposed to the incident light 18a, they may generate a fluorescent signal 18b that is directed towards the photosensitive detector 20. The fluorescent signal may have a wavelength that is greater than the cutoff wavelength of the dichroic mirror 12. Therefore, the fluorescent signal 18b may pass through the dichroic mirror 12. The filter 14 may be a band pass filter adapted to filter out wavelengths other than the wavelength of the fluorescent signal. Alternatively, the filter 14 may permit other selected wavelengths to pass through as well. The fluorescent signal 18b may also pass through an aperture 16 to the imaging lens 10. The imaging lens 10 may focus the fluorescent signal 18b, which corresponds to light emitted from the entire field of view, onto a plurality of pixels 22 of the photosensitive detector 20. In some instances, the fluorescent signal 18b may be focused onto a first portion 28 of the photosensitive detector while second portions 30 of the photosensitive detector are not exposed to the fluorescent signal. However, in some embodiments, the fluorescent signal may be focused onto an entire surface of a photosensitive detector as the disclosure is not so limited.

Depending on the photosensitive detector used and the desired application, the one or more pixels 22 may have any desired size field of view. This may include field of views for individual pixels that are both smaller than and larger than a desired cell size. Consequently, a fluorescent signal 18b emitted from a surgical bed may be magnified or demagnified by the imaging device's optics to provide a desired field of view for each pixel 22, see demagnification in FIG. 1. Additionally, in some embodiments, the optics may provide no magnification to provide a desired field of view for each pixel 22.

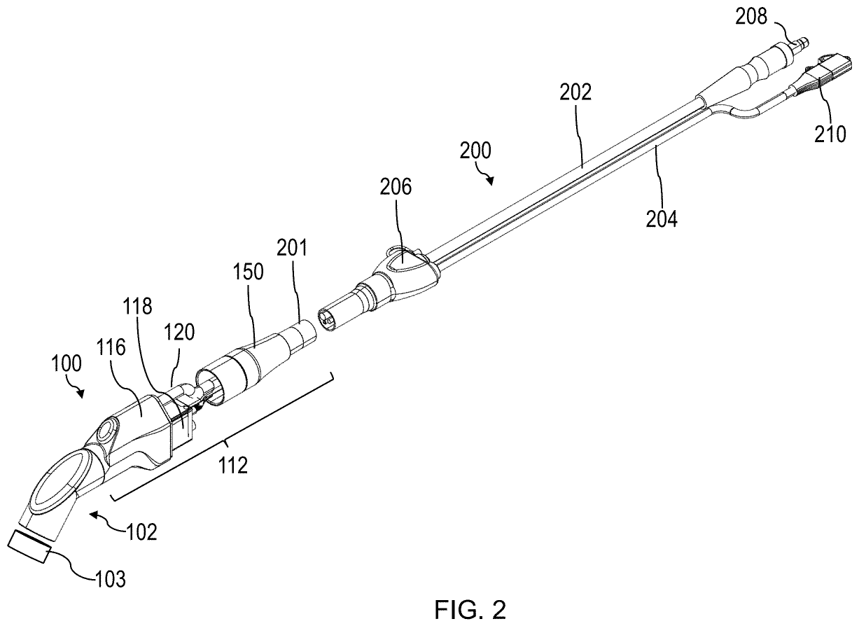

Having generally described embodiments related to a medical imaging device and an associated rigid imaging tip, specific embodiments of a medical imaging device and its components are described in more detail below with regards to FIGS. 2-4B.

FIG. 2 depicts a perspective view of a medical imaging device 100 and hybrid cable 200. As shown in FIG. 2, the imaging device 100 includes a rigid imaging tip 102 configured to be placed on tissue to image the tissue at a focal length set by a distal end of the imaging tip. The imaging device includes a body 112 that may be manipulated by a user (e.g., a surgeon). In some embodiments as shown in FIG. 2, the body of the device includes a housing 116 having a portion that functions as a handle so that the device may be hand operated. The body houses a light guide 120 and a photosensitive detector 118. The light guide 120 may be configured to illuminate the targeted tissue for imaging. In particular, the light guide 120 may be configured to provide an excitation light at a desired wavelength range that excites fluorescence of an imaging agent. As will be discussed further with reference to exemplary embodiments below, the light may pass from the light guide 120 through several reflecting surfaces, lens, filters, and/or other optical elements before reaching the imaging tip 102. The light guide 120 as shown in FIG. 2 is a fiber optic cable, which may be connected to an external light source via the hybrid cable 200. As shown in FIG. 2, the light guide 120 and the photosensitive detector 118 are attached to a housing 116. The housing 116 may house the various optical components. The housing may also include the imaging tip 102. As shown in FIG. 2, the medical imaging device includes a removable tip 103 that may be attached to the imaging tip 102. As will be discussed further below, the removable tip 103 may include a window and may be configured to engage a tissue bed to flatten the tissue bed within a depth of field of the photosensitive detector 118. The housing 116 may also provide a handling surface (e.g., a handle) for a user of the medical imaging device 100. According to some embodiments as shown in FIG. 2, the medical imaging device may also include a tapered housing portion 150 which may assist in sealing the housing 116 from fluid ingress. In some embodiments, the tapered housing portion may compress and seal a portion 201 of the hybrid cable 200 entering the body 112.

According to the embodiment of FIG. 2, the medical imaging device 100 includes a hybrid cable 200. The hybrid cable may function to connect the light guide 120 and the photosensitive detector 118 to an external light source, a power source and/or processor, respectively. As shown in FIG. 2, the hybrid cable includes an optical cable 202 configured to pipe light from an external light source to the light guide 120. The hybrid cable 200 also includes a detector cable 204. In some embodiments, the detector cable 204 may transmit both power and signals from the photosensitive detector in some embodiments. However, instances in which separate cables are used for power and signal transmission are also contemplated. Regardless of the specific arrangement, the detector cable 204 may connect the photosensitive detector 118 to a computing device including one or more processors configured to receive signals from the photosensitive detector. In some embodiments, the detector cable may employ a standardized protocol for data and power, such as USB 2.0, USB 3.0, USB-C, or any other suitable protocol. As shown in FIG. 2, the hybrid cable includes a proximal connector 206 which receives both the optical cable 202 and the detector cable 204. In some embodiments, the proximal cable is configured to provide a waterproof seal between the optical cable and the detector cable. The hybrid cable also includes an optical connector 208 configured to connect to an external light source. The hybrid cable also includes a detector connector 210 configured to connect the detector to an external device (e.g., a computing device). Of course, while a wired medical imaging device 100 is shown including a hybrid cable 200 in the embodiment of FIG. 2, in other embodiments data may be transmitted wirelessly to an external device (e.g., a computing device). For example, the medical imaging device 100 may include a wireless transmitter or transceiver configured to send or receive information from an external device (e.g., a computing device). In some embodiments, a medical imaging device 100 may be wired to a light source and power source but may transmit information wirelessly to an external device having one or more processors. Of course, any suitable combination of wired and wireless connections may be employed, as the present disclosure is not so limited.

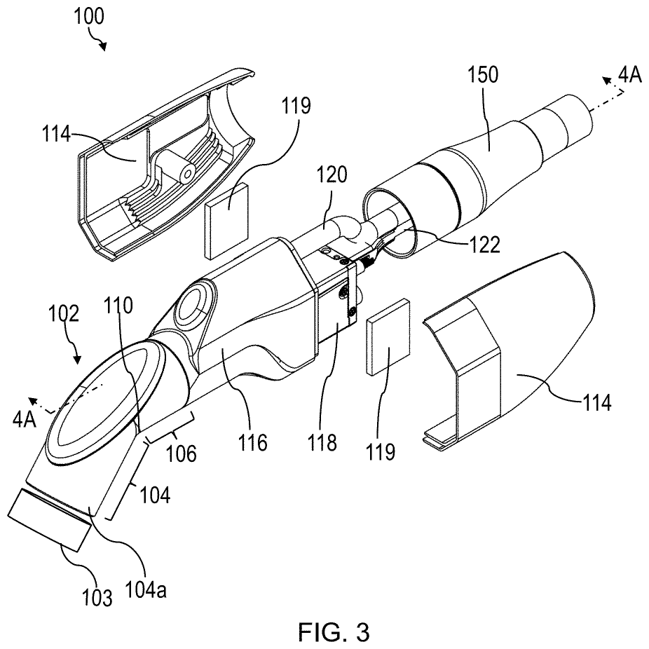

FIG. 3 depicts a partially exploded view of a medical imaging device 100 including a distally extending rigid imaging tip 102. The rigid imaging tip 102 may include a distal portion 104 and a proximal portion 106. A distal end 104a of the rigid imaging tip located on the distal portion 104 may at least partly define a field of view for the imaging device. In some embodiments, the proximal portion 106 may be constructed to either be detachably or permanently connected to a housing 116 of the imaging device. In some embodiments, the rigid imaging tip may also be made from materials that are compatible with typical sterilization techniques such as various steam, heat, chemical, and radiation sterilization techniques.

As shown in FIG. 3, the medical imaging device 100 includes a removable tip 103 configured to be removably attached to the distal end 104a of the rigid imaging tip 102. The removable tip may be configured to protect the rigid imaging tip during use of the device with a tissue bed. In some embodiments, the removable tip 103 may include one or more optically transparent windows configured to allow light to pass through the removable tip. In some embodiments, the removable tip may be configured to be pressed against a tissue bed to flatten the tissue within a depth of field of a photosensitive detector 118. In some embodiments, the connection between the rigid imaging tip 102 and the removable tip 103 may include, for example, a snap on, screw on, suction, magnetic connection, and/or any other appropriate type of connection. This may provide multiple benefits including, for example, easily and quickly changing a rigid imaging tip during a surgical procedure as well as enabling the rigid imaging tip to be removed and sterilized. In some embodiments, the removable tip 103 may be removed from the medical imaging device after use.

In some embodiments as shown in FIG. 3, the housing 116 of the medical imaging device 100 may include a light guide covering portion 114. As shown in FIG. 3, the housing 116 is configured to mount the photosensitive detector 118 to the medical imaging device. The light guide covering portion 114 houses thermal pads 119 configured to absorb heat from the photosensitive detector. In some embodiments, the light guide covering portion 114 may be configured to cover the light guide 120 and the photosensitive detector 118. In some embodiments, the photosensitive detector 118 may include an appropriate data output 122 for outputting data to an external device (e.g., a computing device). In some embodiments, the data output may include a detector cable, as described previously with reference to FIG. 2. Additionally, in some embodiments, the photosensitive detector may include a power input. In some embodiments, the power input may include a detector cable, as described previously with reference to FIG. 2. In some embodiments, the data output 122 may include an integrated power input to the photosensitive detector 118, for example, in the form of a detector cable (see FIG. 2, for example). In some embodiments, one or more light guides 120 associated with one or more separate light sources, not depicted, may be covered by the light guide covering portion 114. As discussed previously the light guide 120 may provide light including at least a first range of excitation wavelengths to the medical imaging device 100. According to the embodiment of FIG. 3, the medical imaging device includes a tapered housing portion 150 configured to compress and seal any cable(s) entering the housing 116.

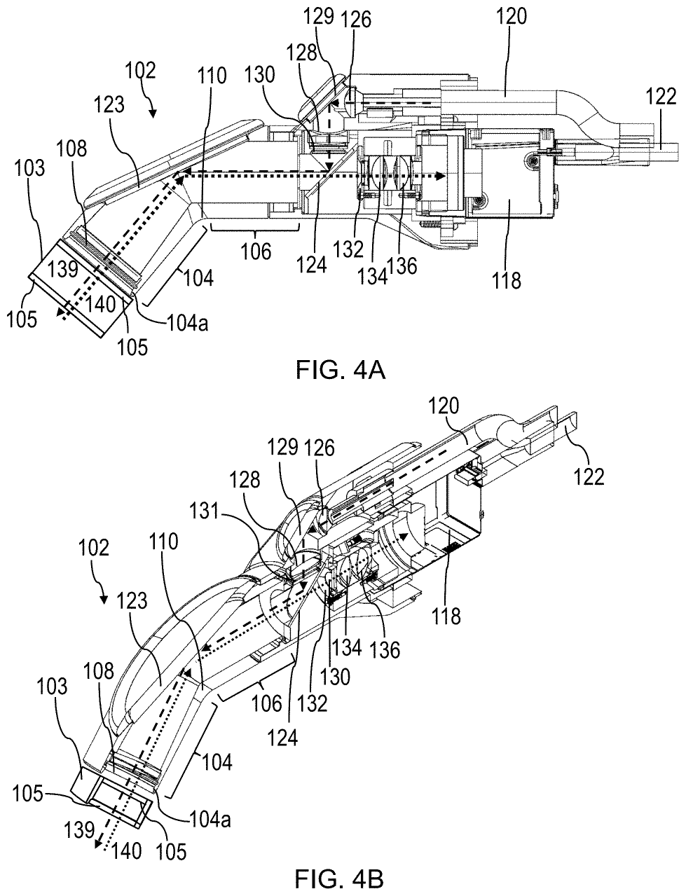

FIGS. 4A-4B depict cross sectional views of the medical imaging device of FIG. 3 taken along line 4A-4A. The cross sections of FIGS. 4A-4B depict the optical arrangement of the medical imaging device. As shown in FIGS. 4A-4B, the medical imaging device includes a rigid imaging tip 102 corresponding to a member distally extending from the housing 116 with an optically transparent or hollow interior. A distal end 104a of the rigid imaging tip 102 may define a focal plane located at a fixed distance relative to the optically coupled photosensitive detector 118 located on a proximal portion of the medical imaging device. In one embodiment, the optics coupling the rigid imaging tip and the photosensitive detector may include an objective lens 134 and an imaging lens 136 located between the rigid imaging tip and the photosensitive detector. The objective and imaging lenses 134 and 136 may focus light emitted from within a field of view of the rigid imaging tip onto a surface of the photosensitive detector 118 including a plurality of pixels. A magnification or demagnification provided by the combined objective and imaging lenses 134 and 136 may be selected to provide a desired field of view for each pixel.

As shown in FIGS. 4A-4B, the medical imaging device 100 may also include one or more dichroic mirrors 124 located between the photosensitive detector 118 and a distal end 104a of the rigid imaging tip. The dichroic mirror 124 may be adapted to reflect light below a cutoff wavelength towards the distal end of the rigid imaging tip and transmit light above the cutoff wavelength towards the photosensitive detector 118. In the current embodiment, the cutoff wavelength may be greater than an excitation wavelength of a desired imaging agent and less than an emission wavelength of the imaging agent. While any appropriate structure might be used for the dichroic mirror, in one embodiment, the medical imaging device includes a single dichroic mirror along an optical path of the medical imaging device.

In some embodiments as shown in FIGS. 4A-4B, the medical imaging device 100 may include one or more filters 130 located between the dichroic mirror 124 and the photosensitive detector 118. The one or more filters 130 may be adapted to permit light emitted from an imaging agent to pass onto the photosensitive detector while blocking light corresponding to excitation wavelengths of the imaging agent. Depending on the embodiment, the one or more filters may either permit a broad spectrum of wavelengths to pass or they may only permit the desired excitation wavelength, or a narrow band surrounding that wavelength, to pass as the disclosure is not so limited.

In some embodiments as shown in FIG. 4A-4B, an aperture stop 132 including an appropriately sized aperture may also be located between the rigid imaging tip 102 and the photosensitive detector 118. More specifically, the aperture stop 132 may be located between the dichroic mirror 124 and the imaging lens 136. Depending on the embodiment, the aperture may have an aperture diameter selected to provide a desired f number, depth of field, and/or reduction in lens aberrations. Appropriate aperture diameters may range from about 5 mm to 15 mm inclusively which may provide an image side f number between about 3 to 3.5 inclusively. However, other appropriate aperture diameters and f numbers are also contemplated.

During use of the medical imaging device 100, the light guide 120 may receive light from an associated light source. The light guide 120 may be any appropriate structure including, for example, fiber-optic cables used to transmit light from the associated light source to the medical imaging device. According to the embodiment of FIGS. 4A-4B, the light guide 120 is configured to extend in a direction that is parallel to a longitudinal axis of a portion of the medical imaging device the light guide extends through. Accordingly, as shown in FIGS. 4A-4B, the light guide 120 is orientated parallel to the direction of imaging of the photosensitive detector 118 along an associated portion of the optical path though other orientations of these components may also be used as the disclosure is not so limited. In some embodiments, the light guide 120 may be associated with optics such as an aspheric lens 126 disposed on a distal end of the depicted optical fiber bundle of the light guide 120 to help collimate light directed towards the dichroic mirror 124. As shown in FIGS. 4A-4B, the light guide may also include an additional collimating lens to further collimate light toward the dichroic mirror 124. The light guide 120 may also be optically coupled with one or more filters 131 disposed between the light guide and the dichroic mirror in order to provide a desired wavelength, or a spectrum of wavelengths, to the dichroic mirror 124 and ultimately the rigid imaging tip 102. This wavelength, or spectrum of wavelengths, may correspond to one or more excitation wavelengths of a desired imaging agent used to mark abnormal tissue for imaging purposes. Depending on the embodiment, the light guide 120 may either be associated with a single light source, or it may be associated with multiple light sources. Alternatively, multiple light inputs may be coupled to the medical imaging device to provide connections to multiple light sources as the current disclosure is not so limited.

According to the embodiment of FIGS. 4A-4B, as the light guide 120 is oriented parallel to a longitudinal axis of the of the medical imaging device, the dichroic mirror 124 is not in a direct optical path of the light guide. Accordingly, as shown in FIGS. 4A-4B, the medical imaging device may include a light guide mirror 129 configured to redirect the light from the light guide 120 towards the dichroic mirror 124. That is, the light guide mirror 129 reflects the light from the light guide approximately 90 degrees toward the dichroic mirror 124. In some embodiments as shown in FIGS. 4A-4B, the light guide mirror is disposed between the aspheric lens 126 and the collimating lens 128, though other arrangements are contemplated, as the disclosure is not so limited. The path of light provided by the light guide is shown by light guide path 139, which is discussed further below. While a mirror is employed in the embodiment of FIGS. 4A-4B, in other embodiments other light bending elements may be employed, including, but not limited to, prisms, fiber optics, etc., as the present disclosure is not so limited.

It should be understood that the above components may be provided in any desired arrangement. Additionally, a medical imaging device may only include some of the above noted components and/or it may include additional components. However, regardless of the specific features included, an optical path 140 of a medical imaging device may pass from a distal end 104a of a rigid imaging tip 102 to a photosensitive detector 118. For example, light emitted from within a field of view may travel along an optical path 140 passing through the distal end 104a as well as the distal portion 104 and proximal portion 106 of the rigid imaging tip. The optical path may also pass through the housing 116 including various optics to the photosensitive detector 118.

According to the embodiment of FIGS. 4A-4B, a medical imaging device 100 includes a rigid imaging tip 102 with a distal portion 104 and a proximal portion 106. The distal portion 104 may include a distal end 104a including an opening optically coupled with a photosensitive detector 118. The rigid imaging tip includes a window 108 integrated with the distal end 104a of the rigid imaging tip. The window 108 may be transparent to both the excitation wavelengths provided by an associated light source as well as wavelengths emitted from a desired imaging agent. While any appropriate shape might be used depending on the particular optics and algorithms used, in one embodiment, the window 108 may have a flat shape to facilitate placing tissue at a desired focal plane when it is pressed against a surgical bed. Additionally, as shown in the embodiment of FIGS. 4A-4B, the medical imaging device 100 includes a removable tip 103 configured to be removably attached to the distal end 104a of the rigid imaging tip 102. The removable tip may be configured to protect the rigid imaging tip during use of the device with a tissue bed. The removable tip 103 includes two optically transparent windows 105 configured to allow light to pass through the removable tip. In particular, the windows 105 may be transparent to both the excitation wavelengths provided by an associated light source as well as wavelengths emitted from a desired imaging agent. Of course, while two windows are shown in the embodiment of FIGS. 4A-4B, in other embodiments any suitable number of windows may be employed, as the present disclosure is not so limited. In some embodiments, the removable tip 103 may be configured to be pressed against a tissue bed to flatten the tissue within a depth of field of the photosensitive detector 118. For example, one of the windows 105 may be pressed against the tissue to flatten the tissue against the window. In some embodiments a focal plane of the photosensitive detector may be aligned with a distal window 105 of the removable tip 103, such that tissue pressed against the distal window is within a depth of field of the photosensitive detector. In some embodiments, the connection between the rigid imaging tip 102 and the removable tip 103 may include, for example, a snap on, screw on, suction, magnetic connection, and/or any other appropriate type of connection.

In some embodiments as shown in FIGS. 4A-4B, the rigid imaging tip 102 includes a bend 110 to facilitate access of a medical imaging device into a surgical site. For example, a distal portion 104 of the rigid imaging tip may be angled relative to a proximal portion 106 of the rigid imaging tip. Any appropriate angle between the proximal and distal portions to facilitate access to a desired surgical site might be used. However, in one embodiment, an angle between the proximal and distal portions may be between about 25° to 65°. For example, a rigid imaging tip may have an angle that is equal to about 45°. In embodiments including an angled distal portion, the rigid imaging tip 102 includes a mirror 123 adapted to bend an optical path 140 and light guide path 139 through the bent rigid imaging tip. The mirror may be positioned at the bend 110 of the rigid imaging tip, such that light traveling through the proximal portion 106 is reflected through the distal portion 104. Likewise, light traveling through the distal portion 104 is reflected by the mirror through the proximal portion 106. In this manner the mirror provides a reflective surface allowing for the transmission of both excitation light and light emitted from a desired imaging agent to travel through the rigid imaging tip 102. It should be understood that even though a bent configuration with a mirror 123 is shown in the exemplary embodiment of FIGS. 4A-4B, one or more other light bending components (e.g., prisms, fiber optics, etc.) may be employed, as the present disclosure is not so limited. Additionally, in some embodiments, a straight imaging tip may be employed without any mirror, as the present disclosure is not so limited.

According to the embodiment of FIGS. 4A-4B, the light guide path 139 and optical path 140 are substantially parallel along at least a portion of a length of the imaging device. The optical path 140 originates at the distal end 104a, reflects off the mirror 123 and proceeds through the dichroic mirror 124 to the photosensitive detector. The light guide path originates at the light guide 120, reflects off the light guide mirror 129, reflects off the dichroic mirror 124 toward the rigid imaging tip 102, and finally reflects off the mirror 123 and exits the distal end 104a of the rigid distal tip. Accordingly, the light guide path 139 and optical path 140 are parallel from the dichroic mirror 124 through the distal end 104a of the rigid imaging tip. In some embodiments, a portion of the optical path 140 and a portion of the light guide path 139 are coincident along a length of the imaging device between the dichroic mirror 124 and the distal end 104a. Of course, any suitable optical path and light guide path may be employed in a medical imaging device, as the present disclosure is not so limited.

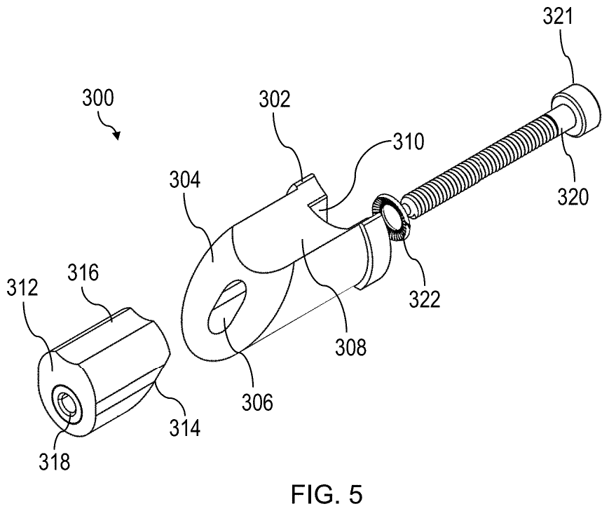

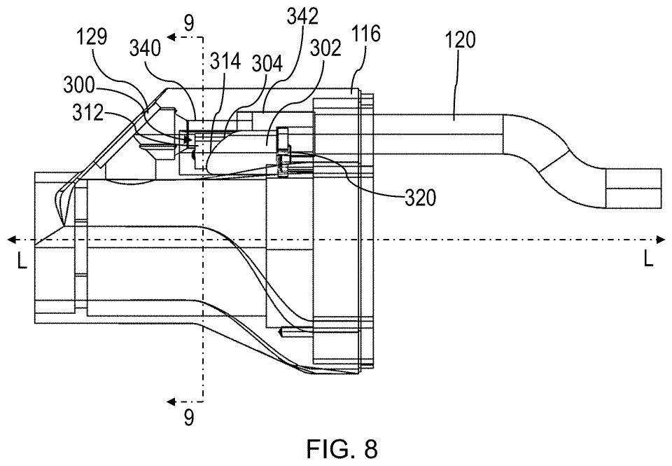

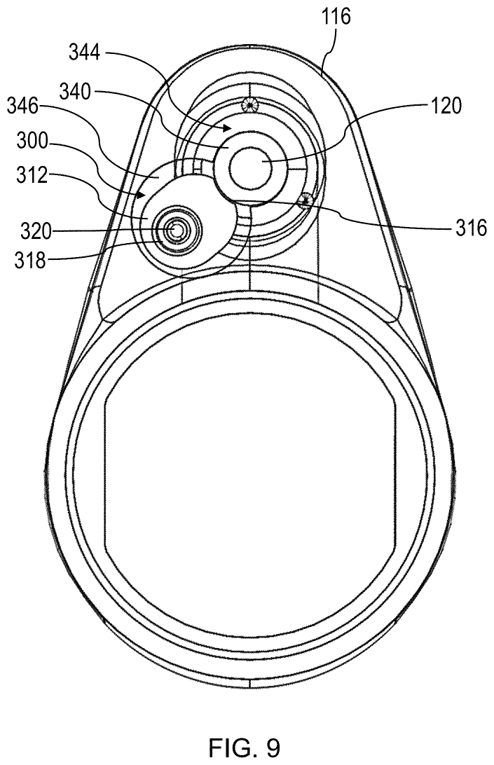

FIG. 5 is a perspective exploded view of one embodiment of a light guide clamp 300 for an imaging device. The clamp 300 may be configured to secure a light guide within a housing of the imaging device. The clamp is configured to apply a force to the light guide to hold the light guide against the housing in a desired position. An exemplary relationship between the clamp 300, a light guide, and housing, will be shown and described further with reference to FIGS. 8-9. As shown in FIG. 5, the clamp includes a clamp body 302, a clamp wedge 312, and an adjustment fastener 320. The clamp body includes a clamp body inclined surface 304 that is inclined relative to a longitudinal axis of the clamp. The clamp wedge includes a clamp wedge inclined surface 314 that is inclined relative to a longitudinal axis of the clamp. In some embodiments as shown in FIG. 5, the angles formed between the clamp body and clamp wedge inclined surfaces and the longitudinal axis of the clamp may be supplementary angles relative to the longitudinal axis of the clamp. In such an embodiments, acute angles formed between the clamp body and clamp wedge inclined surfaces and the longitudinal axis of the clamp may be equal.

According to the embodiment of FIG. 5, the clamp body inclined surface 304 is configured to engage the clamp wedge inclined surface 314 to move the clamp wedge into or out of engagement with a light guide (e.g., between an engaged clamp position and a disengaged clamp position). In particular, the clamp body inclined surface 304 is configured to engage the clamp wedge inclined surface. Accordingly, as the clamp wedge moves relative to the clamp body, the two inclined surfaces will slide along each other, and the clamp wedge will be displaced relative to the clamp body. In particular, when a longitudinal spacing between the clamp wedge and clamp body is adjusted by adjusting a length of the adjustment fastener extending between the clamp wedge and clamp body as elaborated on below, the clamp wedge may move transversely relative to the clamp body to increase a transverse dimension of the overall clamp in at least one direction. In this manner, the adjustment of the longitudinal spacing between the clamp body and the clamp wedge may allow the clamp to apply a traverse force to an adjacent light guide. In some embodiments as shown in FIG. 5, the clamp wedge inclined surface and the clamp body inclined surface may have equal angles relative to a longitudinal axis of the clamp. Of course, the clamp wedge inclined surface and the clamp body inclined surface may have any suitable angle that is the same or different, as the present disclosure is not so limited. Additionally, instances in which one of the surfaces is not inclined and simply corresponds to an appropriately shaped camming surface to permit sliding contact between the camming surface and the corresponding inclined surface are also contemplated. Additionally, instances in which non-linear inclined surfaces are incorporated into a clamp are also contemplated as the disclosure is not limited in this fashion.

According to the embodiment of FIG. 5, the longitudinal spacing of the clamp body 302 and the clamp wedge 312 may be adjusted with the adjustment fastener 320 (e.g., by rotation of the adjustment fastener). In the embodiment of FIG. 5, the adjustment fastener is configured to extend in a direction parallel to a longitudinal axis of the clamp 300. As shown in FIG. 5, the clamp body includes a slot 306 configured receive the adjustment fastener 320. The slot 306 forms a through hole through the clamp body 302. As shown in FIG. 5, the slot extends in a dimension transverse (e.g., perpendicular) to a longitudinal axis of the clamp. Accordingly, when disposed in the slot, the adjustment fastener 320 is able to move transversely relative to the longitudinal axis of the clamp 300. As shown in FIG. 5, the clamp wedge includes a threaded portion 318, such as a threaded through hole, insert, or other feature. The threaded portion is configured to threadedly engage the adjustment fastener. Accordingly, rotation of the adjustment fastener 320 may be employed to adjust a longitudinal spacing between the clamp wedge and the clamp body. For example, threading the adjustment fastener into the threaded portion 318 may move the clamp wedge into further engagement with the clamp body 302. Additionally, threading the adjustment fastener into the threaded portion 318 may reduce an overall distance between a head 321 of the adjustment fastener and the threaded portion 318 (e.g., an effective length of the fastener is decreased). The adjustment fastener may be under tension when threaded into the threaded portion, thereby applying force urging the clamp wedge inclined surface 314 into engagement with the clamp body inclined surface 304. This force is converted into force in a transverse direction relative to the longitudinal axis of the clamp, thereby moving the clamp wedge 312 in the transverse direction. Unthreading the adjustment fastener from the threaded portion 318 may move the clamp wedge into less engagement with the clamp body. Additionally, unthreading the adjustment fastener into the threaded portion 318 may increase an overall distance between a head 321 of the adjustment fastener and the threaded portion 318 (e.g., an effective length of the fastener is increased). The adjustment fastener may be under reduced tension when unthreaded from the threaded portion, allowing the clamp wedge to move radially inward. In this manner, rotation of the adjustment fastener may be employed to move the clamp wedge transversely to apply force to a light guide. As the adjustment fastener 320 is threadedly engaged with the threaded portion, the adjustment fastener may move transversely with the clamp wedge. Accordingly, the traverse spacing provided by the slot 306 allows for this movement. Additionally, in some embodiments, the clamp body includes a head slot 310 configured to accommodate a head 321 of the adjustment fastener and allow for movement of the head with the clamp wedge. In some embodiments as shown in FIG. 5, the adjustment fastener may include a lock washer 322 configured to inhibit unintentional rotation of the adjustment fastener when the adjustment fastener is tightened to apply a force to a light guide.

According to the embodiment of FIG. 5, the clamp body includes a clamp body groove 308. The clamp body groove may be configured to complement the shape of a portion of a light guide. Such an arrangement may spread forces out across the light guide to avoid concentration of stress in a particular area. In some embodiments as shown in FIG. 5, the clamp body groove has a concave shape that may be configured to accommodate a cylindrical portion of a light guide. In some embodiments as shown in FIG. 5, the clamp wedge includes a clamp wedge groove 316. The clamp wedge groove may have a shape complementary to that of distal end portion of a light guide. For example, in the embodiment of FIG. 5, the clamp wedge groove has a concave shape configured to complement a cylindrical or otherwise complementarily curved distal end portion of the light guide. The clamp wedge groove may be configured to spread force out over a wider surface area of the light guide, such that a maximum pressure applied to the light guide may be reduced when compared to a clamp wedge having no complementarily shaped groove.

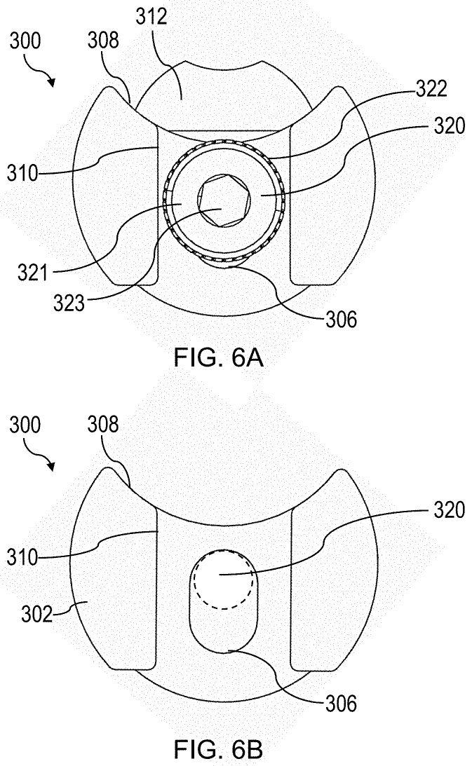

FIG. 6A is a rear view of the light guide clamp 300 of FIG. 5 depicting an adjustment fastener 320 and a head slot 310. As shown in FIG. 6A and discussed previously, the adjustment fastener is disposed in a slot 306 formed through the clamp body 302. The adjustment fastener includes a head 321, which may include a socket 323 as shown in FIG. 6A. A tool (e.g., a hex key) may be employed to apply torque to the adjustment fastener to thread or unthread the adjustment fastener from the clamp wedge 312. As shown in FIG. 6A, the clamp body includes a head slot 310 configured to accommodate the head 321 of the adjustment fastener. As the adjustment fastener is rotated (e.g., loosened or tightened) to adjust a longitudinal spacing between the clamp wedge 312 and the clamp body 302, the adjustment fastener may move transverse to a longitudinal axis of the clamp 300 (e.g., a direction in which the adjustment fastener extends). The head slot 310 allows the head 321 of the adjustment fastener to move without interference.

FIG. 6B a is rear view of a clamp body 302 of the light guide clamp 300 of FIG. 6A with the clamp wedge and adjustment fastener removed for clarity. A phantom adjustment fastener 320 is shown in dashed to represent the physical arrangement between the clamp body and the adjustment fastener. According to the embodiment of FIG. 6B, the slot 306 extends in a transverse (e.g., perpendicular) dimension relative to the longitudinal axis of the clamp 300 (e.g., the slot extends vertical relative to the page). Accordingly, the adjustment fastener is able to slide in the slot 306 in the transverse dimension. When an adjustment fastener is employed to adjust a longitudinal spacing between a clamp wedge and the clamp body to move the clamp wedge transversely, the fastener is able to correspondingly move with the clamp wedge. Of course, the slot 306 may extend in any suitable direction to accommodate movement of the adjustment fastener with the clamp wedge, as the present disclosure is not so limited.

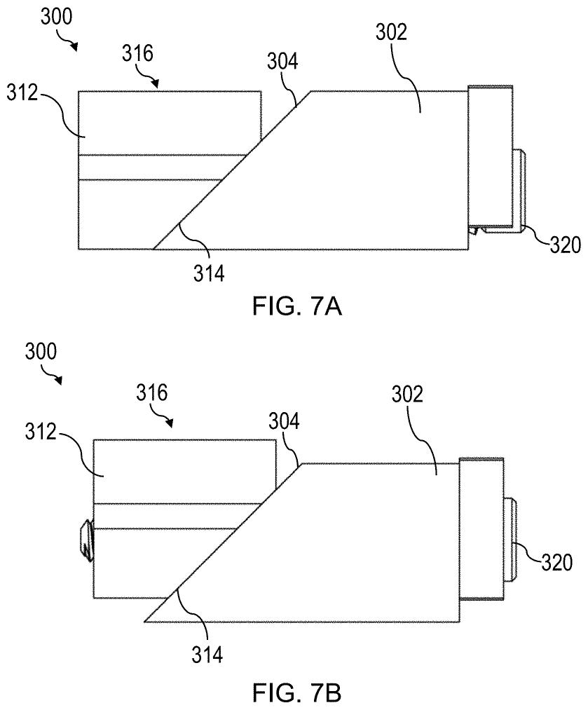

FIG. 7A is a side view of the light guide clamp 300 of FIG. 5 in a first state, and FIG. 7B is a side view of the light guide clamp in a second state. FIGS. 7A-7B show the movement of the clamp wedge 312 and adjustment fastener 320 as a longitudinal spacing between the clamp wedge and the clamp body is adjusted. In the first state of FIG. 7A, the clamp wedge is engaged with a lowermost portion of the clamp body inclined surface 304. The state shown in FIG. 7A may correspond to a disengaged clamp position, as the clamp wedge 312 does not extend transversely past the clamp body 302 relative to a longitudinal axis of the clamp. In the state of FIG. 7A, the adjustment faster may be disposed in a bottom portion of a slot. In the second state of FIG. 7B, the clamp wedge 312 has been moved to an engaged clamp position, where the clamp wedge extends transversely past the clamp body 302 relative to a longitudinal axis of the clamp. Accordingly, the clamp wedge 312 may apply force to a light guide to secure the light guide in a housing. As shown in FIG. 7B, the adjustment fastener has been threaded into the wedge portion and extends longitudinally out of the wedge portion. Accordingly, the clamp wedge has been moved into further engagement with the clamp wedge (e.g., the longitudinal spacing of the clamp wedge and body as well as an overall length of the clamp corresponding to the combined clamp wedge and body has been reduced relative to the first state) and the clamp wedge has moved along the clamp body inclined surface 304. As a result, the clamp wedge has been moved transversely relative to the longitudinal axis of the clamp due to the inclination of the clamp inclined surface. Likewise, as the adjustment fastener is threadedly engaged with the clamp wedge, the adjustment fastener has moved correspondingly with the clamp wedge in the transverse direction. Accordingly, the adjustment fastener 320 may be disposed in an upper portion of the slot formed in the clamp body 302. To move the clamp wedge to the disengaged clamp position, the adjustment fastener may be unthreaded from the clamp wedge to increase the spacing between the clamp wedge and the clamp body. Increasing the spacing between the clamp wedge and the clamp body may allow the clamp wedge to move down the clamp body inclined surface.

FIG. 8 is a side view of one embodiment of an imaging device housing 116 including a light guide clamp 300. As shown in FIG. 8, the light guide may be a fiber optic cable configured to be disposed in the housing. The light guide has a light guide longitudinal axis that is parallel to a longitudinal axis L of the imaging device housing 116 along a distal portion of the light guide. In some embodiments, the light guide longitudinal axis may be parallel to an optical path of the imaging device within the housing 116. In some embodiments, an optical path and a longitudinal axis L may be parallel. As shown in FIG. 8, the light guide includes a rigid exterior portion 340 disposed on a distal end portion of the light guide, which in the embodiment of FIG. 8 is configured as a rigid jacket. The rigid exterior portion is configured to protect internal components of the light guide (e.g., optical fibers) from the force applied by the clamp 300. The light guide also includes a flexible protective coating 342 which is configured to provide general protection for the light guide. For example, the protective coating may be formed as a polymer sheath extending along a length of the depicted fiber optic cable.

According to the embodiment of FIG. 8, the light guide 120 is configured to be secured to the housing 116 in a desired position by the clamp 300. In particular, the clamp is configured to apply force to the rigid exterior portion 340 against the housing 116 to fix the light guide in place. In this manner, in some embodiments the clamp may function to fix a position and orientation of the clamp and light guide relative to the housing of the imaging device. As shown in FIG. 8, the clamp includes a clamp body 302, a clamp wedge 312, and an adjustment fastener 320. Like previously discussed embodiments, the clamp body includes a clamp body inclined surface, and the clamp wedge includes a clamp wedge inclined surface configured to engage the clamp body inclined surface. The adjustment fastener is configured to adjust a longitudinal spacing of the clamp wedge and clamp body (e.g., along axis L), which is converted into movement of the clamp wedge in a clamping direction transverse to the longitudinal axis (e.g., perpendicular to the longitudinal axis). Moving the clamp wedge in the clamping direction may move the clamp wedge into contact with the rigid exterior portion 340 of the light guide such that the clamp applies force to the rigid exterior portion. In some embodiments, the clamp body 302 may be retained in a channel such that the longitudinal axis of the clamp body is fixed relative to the longitudinal axis of the housing 116. An exemplary arrangement of channels of the housing is described further with reference to FIG. 9. In some embodiments as shown in FIG. 8, the adjustment fastener may be accessible to a tool in a direction parallel to the longitudinal axis L.

FIG. 9 is a cross-sectional view of the imaging device housing 116 of FIG. 8 taken along line 9-9. According to the embodiment of FIG. 9, the clamp 300 is positioned adjacent to the light guide 120. In some embodiments as shown in FIG. 9, the light guide is positioned between the clamp 300 and a supporting portion of the housing 116. Accordingly, in some embodiments, the clamp 300 is configured to apply a compressive force to the cable against the supporting portion of the housing. In the embodiment of FIG. 9, the light guide 120 and rigid exterior portion 340 are disposed in a first channel 344 of the housing 116. The first channel 344 may be a supporting portion of the housing configured to resist movement of the light guide, and in particular the rigid exterior portion 340, such that the clamp may apply a compressive force to the light guide to secure the light guide to the housing. The first channel 344 is sized and shaped to accommodate sliding of the light guide in the housing 116 along a longitudinal axis of the housing, and, in some embodiments, parallel to an optical path through the housing. However, the first channel is sized and shaped to inhibit rotation of the light guide, such that the longitudinal axis of the light guide is fixed relative to the housing 116. In some embodiments, the first channel may have a shape approximately equivalent to an exterior shape of the light guide 120. For example, in the embodiment of FIG. 9, the first channel 344 and the light guide 120 have cylindrical shapes. Accordingly, the first channel may resist movement of the light guide in any direction that is no along a shared longitudinal axis of the first channel and the light guide. The rigid exterior portion 340 is configured to engage the first channel under force from the clamp 300 to secure the light guide to the housing. Of course, while a cylindrical first channel and light guide are employed in the embodiment of FIG. 9, in other embodiments any suitable shape may be employed, as the present disclosure is not so limited.

In some embodiments, a clamp of an imaging device may be disposed in a cavity of an imaging device housing. The cavity may be configured to secure the clamp in the housing in a desired position and orientation. In some embodiments, the cavity may have a shape corresponding to the shape of a portion of the clamp, such that the cavity resists movement of the clamp from the desired position and orientation. In some embodiments, the cavity is configured to allow a transverse dimension of the clamp to be adjusted (e.g., by adjusting a fastener as described with reference to exemplary embodiments herein). In some embodiments, the cavity is configured to allow a clamp wedge to move transversely relative to a longitudinal axis of a light guide disposed adjacent the clamp. In some embodiments, the cavity may be at least partially open to a channel of the housing containing the light guide. Accordingly, a clamp may engage the light guide through the cavity and channel to apply force to the light guide. In some embodiments, the cavity and the channel may be at least partially overlapping along at least a portion of their length to allow a portion of the clamp (e.g., a clamp body or clamp wedge depending on the embodiment) to be compressed against the light guide. In some embodiments, the cavity may be a second channel having a channel axis parallel to the first channel containing the light guide. One such exemplary embodiment is discussed further below with reference to FIG. 9.

As shown in FIG. 9, the light guide 120 is disposed in the first channel 344 and the clamp 300 is disposed in a second channel 346 of the housing 116. The second channel 346 may be sized and shaped to retain the clamp. The second channel is also configured to resist movement of the clamp from the desired position and orientation. In some embodiments, the second channel 346 may be sized and shaped to fix a longitudinal axis of the clamp relative the housing and the first channel 344 may be sized and shaped to fix a longitudinal axis of the light guide in place relative the housing. Accordingly, the longitudinal axis of the light guide and the clamp may be fixed relative to one another, such that there is a constant transverse spacing between the longitudinal axes of the clamp and light guide. For example, the second channel 346 may be engaged by a clamp body (see FIG. 8) to fix the longitudinal axis of the clamp relative to the housing. In some embodiments as shown in FIG. 9, the first channel 344 and the second channel 346 are parallel to one another (e.g., extend along parallel axes). According to the embodiment of FIG. 9, the first channel 344 has a first channel axis and the second channel has a second channel axis that are offset from one another. As shown in FIG. 9, the first channel and the second channel are configured to overlap along at least a portion of their overall lengths, such that at least a portion of the first channel is open to the second channel. The clamp 300 is configured to engage the rigid exterior portion 340 of the light guide 120 through the opening between the first channel and the second channel. In some embodiments, a clamp wedge 312 may be configured to extend into the first channel 344 to engage the rigid exterior portion 340. In other embodiments as shown in FIG. 9, the rigid exterior portion 340 may extend into the second channel 346 so that the clamp wedge may engage the rigid exterior portion in the second channel. The clamp wedge is configured to apply force to the rigid exterior portion 340 against the first channel 344 in a transverse clamping direction relative to a longitudinal axis of the light guide to fix the position of the light guide in the first channel. Of course, the clamp wedge may engage the rigid exterior portion in any suitable channel or plurality of channels, as the present disclosure is not so limited. Additionally, while in some embodiments at least a portion of the first channel and second channel may overlap, in other embodiments the first channel and second channel may be spaced from one another and interconnected with one or more transverse openings, as the present disclosure is not so limited.

As shown in FIG. 9, the clamp wedge includes a clamp wedge groove 316 having a shape complementary to a shape of the rigid exterior portion 340. In particular, the clamp wedge groove 316 is concave, and the rigid exterior portion 340 is circular. Accordingly, as shown in FIG. 9, the shapes complement each other when the clamp wedge groove is displaced to engage with the rigid exterior portion of the light guide. Accordingly, when force is applied to the rigid exterior portion with the groove, the force may be spread across a region of the rigid exterior portion.

FIG. 10A is a schematic of one embodiment of a focus of a light guide at a target focal plane 370, and FIG. 10B is a schematic of another embodiment of a focus of a light guide at the target focal plane. The two states shown in FIGS. 10A-10B may be representative of the light emitted from a distal end of an imaging device that originates from a light guide such as a fiber optic cable. The target focal plane 370 may be aligned with a distal end, or other desired focal plane, of an imaging device, as the target tissue for illumination and imaging may be configured to contact the distal end of the imaging device. As shown in FIG. 10A, when the light from a fiber optic light guide is focused on a plane, the illumination may appear as a series of point sources 372. That is, the illumination of the total area of the target focal plane may not be uniform and may instead be concentrated in regions aligned with individual optical fibers. In the state shown in FIG. 10A, a plane of focus of the light guide may be aligned with a focal plane of the imaging device. In contrast, as shown in FIG. 10B, the same light guide but defocused at the target focal plane has a series of blurred sources 374. Accordingly, the light at the focal plane is far more uniform, as the light covers a greater portion of the area of the target focal plane. In the state shown in FIG. 10B, a plane of focus of the light guide may be offset from a focal plane of the imaging device by a predetermined distance. In some cases, more uniformity in illumination at the target focal plane may be desired, at the expense of a maximal illumination intensity that would be provided by a focused light guide. Accordingly, in some embodiments, a method of assembling an imaging device may include first focusing the light emitted from a light guide on the target focal plane, and subsequently adjusting a position of the light guide to be a predetermined distance away from a focal length to the focal plane of the system to defocus light emitted from the light guide on the target focal plane. In this manner, a more uniform illumination may be achieved at the target focal plane for a photosensitive detector. In some embodiments, an imaging device may include a light guide fixed in a position (e.g., by a clamp) such that the light emitted from the light guide is at least partially defocused at a target focal plane of a photosensitive detector.

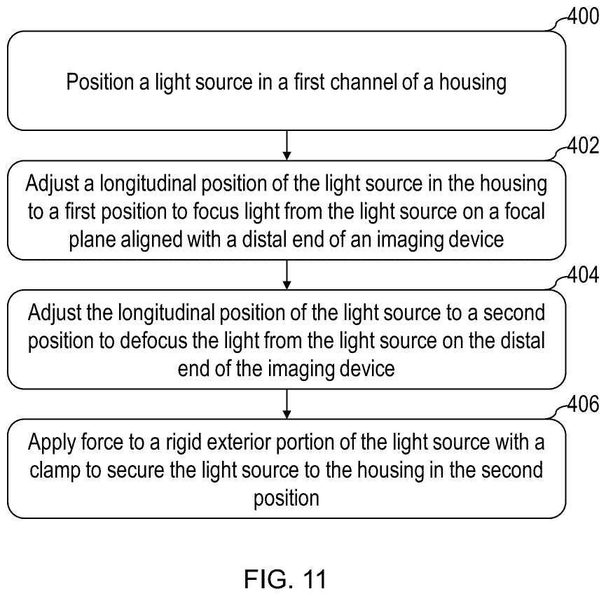

FIG. 11 is a flow chart for one embodiment of assembling an imaging device. In block 400, a light guide is positioned in a first channel of a housing. In some embodiments, positioning the light guide includes sliding the light guide into the first channel along a longitudinal axis of the light guide. In block 402, a longitudinal position of the light guide is adjusted to a first position to focus light from the light guide on a focal plane of the imaging device aligned with a distal end of an imaging device. In some embodiments, focusing the light from the light guide on the focal plane may include moving a light guide focal plane into a depth of field of the imaging device. In some embodiments, adjusting a position of the light guide may include sliding the light guide within the first channel along a longitudinal axis of the light guide. In block 404, a longitudinal position of the light guide is adjusted to a second position to defocus the light from the light guide on the distal end of the imaging device. In some embodiments, defocusing the light from the light guide on the distal end may include moving a light guide focal plane out of a depth of field of the imaging device. In block 406, force is applied to a rigid exterior portion of the light guide with a clamp to secure the light guide to the housing in the second position.

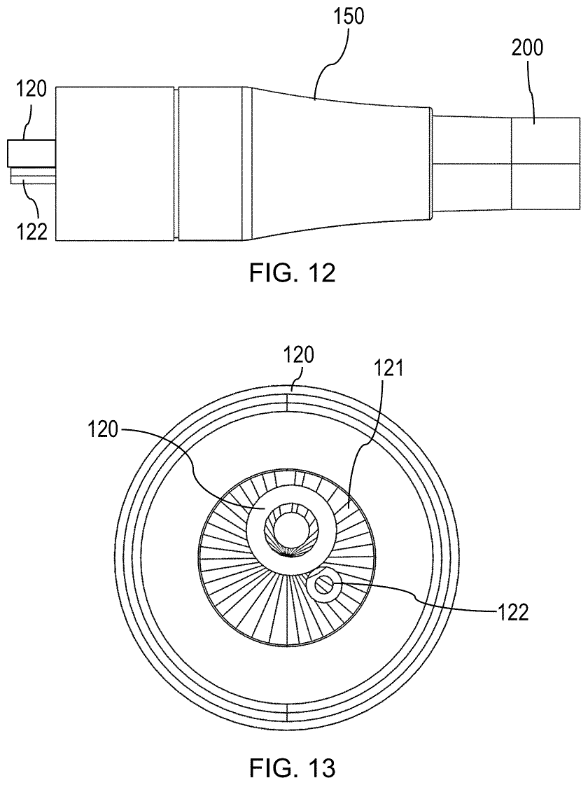

FIG. 12 is a side view and FIG. 13 is a front view of one embodiment of a tapered housing portion 150. As shown in FIG. 12, the taper housing portion is configured to accommodate a light guide 120 and a data output 122. The tapered housing portion is configured to transition the light guide and data output into a hybrid cable 200 as described previously. As shown in FIG. 13, the tapered housing portion includes a strain relief plug 121 configured as an epoxy plug. The strain relief plug may fix the light guide 120 to the tapered housing portion at the strain relief plug. The strain relief plug may also seal a proximal portion of the housing.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. An imaging device comprising:
a housing including a first channel;
a light guide disposed at least partially in the first channel, wherein the light guide includes a rigid exterior portion on a distal end portion of the light guide, and wherein the light guide has a light guide longitudinal axis; and
a clamp disposed in the housing, wherein the clamp is configured to apply a force to the rigid exterior portion of the light guide in a clamp direction transverse to the light guide longitudinal axis to secure the light guide to the housing.

2. The imaging device of claim 1, wherein the housing includes a second channel, wherein the clamp is disposed in the second channel, and wherein the first channel and second channel are at least partially overlapping.

3. The imaging device of claim 2, wherein the first channel has a first channel axis, the second channel has a second channel axis, and wherein the first channel axis is parallel to the first channel axis, and wherein the first channel axis is offset from the second channel axis.

4. The imaging device of claim 1, wherein the light guide is a fiber optic cable.

5. The imaging device of claim 1, wherein the rigid exterior portion is a jacket formed of metal.

6. The imaging device of claim 1, wherein the light guide longitudinal axis is parallel to an optical path through a proximal portion of the housing.

7. The imaging device of claim 1, wherein the clamp includes a clamp body, a clamp wedge, and an adjustment fastener, wherein the adjustment fastener is configured to adjust a longitudinal spacing between the clamp body and the clamp wedge, and wherein the clamp wedge is configured to move transverse to the light guide longitudinal axis when the longitudinal spacing between the clamp body and the clamp wedge is adjusted.

8. The imaging device of claim 7, wherein the clamp wedge includes a wedge inclined surface, wherein the clamp body includes a body inclined surface engaged with the wedge inclined surface.

9. The imaging device of claim 7, wherein the clamp body includes a slot configured to allow the adjustment fastener to move transverse to the light guide longitudinal axis when the longitudinal spacing between the clamp body and the clamp wedge is adjusted.

10. The imaging device of claim 7, wherein the clamp wedge includes a groove having a shape complementing a shape of the rigid exterior portion.

11. The imaging device of claim 1, wherein the clamp direction is perpendicular to the light guide longitudinal axis.

12. The imaging device of claim 1, further comprising a mirror disposed in the housing positioned in an optical path of the light guide, wherein the mirror is configured to reflect light from the light guide in a direction transverse to the light guide longitudinal axis.

13. The imaging device of claim 12, further comprising a dichroic mirror, wherein the mirror is configured to reflect light from the light guide toward the dichroic mirror.

14. The imaging device of claim 1, wherein a focus of the light guide is located at a plane offset by a predetermined distance from a focal plane of the imaging device.

15. The imaging device of claim 14, wherein the focus of the light guide is adjustable by moving the light guide within the housing along the light guide longitudinal axis.

16. An imaging device comprising:
a housing including a first channel;
a light guide disposed at least partially in the first channel, wherein the light guide includes a rigid exterior portion on an exterior distal end portion of the light guide, and wherein the light guide has a light guide longitudinal axis; and
a clamp disposed in the housing, wherein the clamp has a clamp longitudinal axis parallel to the light guide longitudinal axis, and wherein the clamp is configured to apply a force to the rigid exterior portion to secure the light guide to the housing.

17. The imaging device of claim 16, wherein the housing includes a second channel, wherein the clamp is disposed in the second channel, and wherein the first channel and second channel are at least partially overlapping.

18. The imaging device of claim 17, wherein the first channel has a first channel axis, the second channel has a second channel axis, and wherein the first channel axis is parallel to the first channel axis.

19. The imaging device of claim 16, wherein the clamp includes an adjustment fastener configured to receive an adjustment tool in an adjustment direction parallel to the clamp longitudinal axis.

20. The imaging device of claim 16, wherein the light guide is a fiber optic cable.

21. The imaging device of claim 16, wherein the rigid exterior portion if a jacket formed of metal.

22. The imaging device of claim 16, wherein the light guide longitudinal axis is parallel to an optical path through a proximal portion of the housing.

23. The imaging device of claim 16, wherein the clamp includes a clamp body, a clamp wedge, and an adjustment fastener, wherein the adjustment fastener is configured to adjust a longitudinal spacing between the clamp body and the clamp wedge, and wherein the clamp wedge is configured to move transverse to the light guide longitudinal axis when the longitudinal spacing between the clamp body and the clamp wedge is adjusted.

24. The imaging device of claim 23, wherein the clamp wedge includes a wedge inclined surface, wherein the clamp body includes a body inclined surface engaged with the wedge inclined surface.

25. The imaging device of claim 23, wherein the clamp body includes a slot configured to allow the adjustment fastener to move transverse to the light guide longitudinal axis when the longitudinal spacing between the clamp body and the clamp wedge is adjusted.

26. The imaging device of claim 23, wherein the clamp wedge includes a groove having a shape complementing a shape of the rigid exterior portion.

27. The imaging device of claim 16, further comprising a mirror disposed in the housing positioned in an optical path of the light guide, wherein the mirror is configured to reflect light from the light guide in a direction transverse to the light guide longitudinal axis.

28. The imaging device of claim 27, further comprising a dichroic mirror, wherein the mirror is configured to reflect light from the light guide toward the dichroic mirror.

29. The imaging device of claim 16, wherein a focus of the light guide is located at a plane offset by a predetermined distance from a focal plane of the imaging device.

30. The imaging device of claim 29, wherein the focus of the light guide is adjustable by moving the light guide within the housing along the light guide longitudinal axis.

31. A method of assembling an imaging device, the method comprising:
positioning a light guide in a first channel of a housing;
adjusting a longitudinal position of the light guide in the housing from a first position to a second position; and
applying force to a rigid exterior portion of the light guide with a clamp to secure the light guide to the housing in the second position.

32. The method of claim 31, wherein in the first position light from the light guide is focused on a focal plane of the imaging device, and wherein in the second position the light from the light guide is defocused on the focal plane of the imaging device.

33. The method of claim 32, wherein the focal plane is aligned with a distal end of the imaging device.

34. The method of claim 32, wherein adjusting the longitudinal position of the light guide to the second position includes moving a focus of the light guide away from the focal plane of the imaging device.

35. The method of claim 31, wherein the light guide has an optical path through a proximal portion of the housing, wherein positioning the light guide in the first channel includes making a light guide longitudinal axis parallel to the optical path through the proximal portion of the housing.

36. The method of claim 31, wherein applying force to the rigid exterior portion of the light guide includes applying the force in a clamp direction transverse to a light guide longitudinal axis.

37-38. (canceled)

39. The method of claim 38, wherein adjusting the longitudinal spacing between the clamp body and the clamp wedge includes engaging a wedge inclined surface of the clamp wedge with a body inclined surface of the clamp body.

40. The method of claim 39, wherein adjusting the longitudinal spacing between the clamp body and the clamp wedge includes moving the adjustment fastener transverse to the light guide longitudinal axis in a slot formed in the clamp body.

41-42. (canceled)

43. The method of claim 31, wherein positioning the light guide in the first channel includes orienting the light guide toward a mirror disposed in the housing, wherein the method further comprises reflecting light from the light guide off the mirror in a direction transverse to a light guide longitudinal axis.

44. The method of claim 43, wherein reflecting light from the light guide off the mirror includes reflecting light from the light guide toward a dichroic mirror disposed in the housing.
